(19) United States
(12) Patent Application Publication
(10) Pub. No.: US 2019/0117328 A1
(43) Pub. Date: Apr. 25, 2019

(54) Sterile Barrier Packaging Optimized Construction With Plant Based Material

(71) Applicant: NewGen Surgical, Inc., San Rafael, CA (US)

(72) Inventors: Robert N. Chase, San Rafael, CA (US); Kenneth Blier, San Rafael, CA (US); Peter Szyperski, San Rafael, CA (US)

(73) Assignee: NewGen Surgical, Inc., San Rafael, CA (US)

(21) Appl. No.: 16/166,869

(22) Filed: Oct. 22, 2018

Related U.S. Application Data
(60) Provisional application No. 62/574,860, filed on Oct. 20, 2017.

(51) Int. Cl. A61B 50/33; B65D 1/36; B65D 25/34

(52) U.S. Cl. CPC A61B 50/33; A61B 2050/005; B65D 25/34; B65D 1/36

(57) ABSTRACT

The current invention combines use of existing package themoforming, either wet, dry or injection molding to create a base tray for the medical device or products. This base is made from plant based material such as Bamboo, Bagasse, Tapioca and or other sustainable plant based materials. The inside and or the inside and outside of the Base tray is then coated with a film that is either plant based or petroleum based. This film coating creates a functional microbial barrier preventing bacterial from moving through from the outside of the base tray to the inside of the base tray where the sterile medical device or surgical product is housed, The base tray (bottom) can then be used with a either paper or Tyvek or other materials lidding (top) that will be sealed to the base tray for the purposes product sterilization.

### CROSS-REFERENCE TO RELATED APPLICATIONS

This application claims the benefit and priority of U.S. patent application 62/574,860 filed on Oct. 20, 2017.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever. Such trademark(s) may include, “Sterile Barrier Packaging Optimized Construction With Plant Based Material.”

### BACKGROUND OF THE INVENTION

### (1) Field of the Invention

The invention generally relates to packaging systems for medical devices. More particularly, the invention relates to means and methods of fabricating, molding, constructing and sealing sterile barrier systems, using plant based products, to contain and protect medical devices and/or surgical products.

### (2) Description of the Related Art

The known related art fails to anticipate or disclose the principles of the present invention.

In the related art, many traditional plastic packaging systems are known. In the related art, packaging for medical devices and surgical products are made from petroleum based plastic materials.

Traditional petroleum based plastic packaging materials are used in the millions throughout the United States and worldwide. In the related art, medical device packaging is utilized for two primary purposes:

1. Protecting the contents of the packaging from physical damage from its manufacturing point to its point of use; and

2. Acting as a sterile barrier system to allow for sterilization of the contents of the package while acting as a microbial barrier to keep bacteria from contaminating the contents of the package.

While the prior art of medical device packaging is effective at serving the two primary purposes, the prior art has significant shortfalls in creating millions of tons of plastic waste. This plastic packaging waste is sometimes recycled but in many cases is burned or discarded to landfills creating plastic pollution which is increasingly threatening to the environment. Petroleum based packaging is thought to remain in the environment for up to 500 years or longer and persists in the environment for generations.

Plant based packaging material has been used previously as packaging material for items such as eggs, consumer electronics, etc. but has not been used for sterile medical devices. While the prior art plant based packaging would possibly fulfill the first purpose of medical device packaging, to protect the contents from physical damage, in its current state, it would fail the second purpose which is to provide a sterile barrier system.

Thus, there is a significant and urgent need in the art for the present invention.

### BRIEF SUMMARY OF THE INVENTION

The present invention overcomes shortfalls in the related art by presenting an unobvious and unique combination and configuration of methods and components to construct a sterile barrier medical device packaging made from and optimized for plant based material.

The presently disclosed embodiments present a unique and unobvious application and combination of plant based materials to create a sterile barrier packaging that overcomes the existing shortfalls of the prior art of plastic packaging, as the presently disclosed embodiments eschew the use of plastic and other man made substances.

Unique

The current invention combines use of existing package thermoforming, either wet, dry or injection molding to create a base tray for the medical device or products. This base is made from plant based material such as bamboo, bagasse, tapioca and or other sustainable plant based materials. The inside and/or the inside and outside of the base tray is then coated with a film that is either plant based or petroleum based. This film coating creates a functional microbial barrier preventing bacterial from moving from the outside of the base tray to the inside of the base tray where the sterile medical device or surgical product is disposed. The base tray (bottom) can then be used with a either paper or Tyvek or other materials lidding (top) that will be sealed to the base tray for the purposes of product sterilization.

Unobvious

While the disclosed embodiments utilize existing packaging technology used in other industries, the disclosed embodiments artfully combine the existing packaging with a coating process and a microbial barrier lidstock to create a new, unique and unobvious invention.

Useful

By shifting from a traditional petroleum based packaging material (current state of the art-plastic) to a sustainably derived plant based packaging, tons of plastic pollution will be avoided on an annual basis. Creating a sterile barrier packaging system that is optimized for construction with plant based material will not only help the healthcare industry move to a more sustainable future but is will also reduce plastic waste and pollution that threatens the environment and allow healthcare facilities to move from products packaged in a petroleum based system to a renewable, sustainable plant based feedstock.

These and other objects and advantages will be made apparent when considering the following detailed specification when taken in conjunction with the drawings.

### BRIEF DESCRIPTION OF THE DRAWINGS

FIG. 1 depicts a perspective view of a disclosed base

FIG. 2 depicts a perspective view of a lid or cover

FIG. 3 depicts a perspective view of molded pulp fiber trays

FIG. 4 depicts a perspective view of molded pulp fiber with sheet barrier on roll with adhesive

FIG. 5 depicts a disclosed embodiment after undergoing a forming process, conforming to an inner shape of a container

FIG. 6 depicts a top view of a disclosed embodiment

FIG. 7 depicts a single tray with film prior to a die cut

FIG. 8 depicts a single tray with film after a die cut

FIG. 9 depicts a side view of a tray with a sheet barrier

FIG. 10 depicts a product within a disclosed tray

FIG. 11 depicts a gas permeable barrier lid covering a product disposed within a tray

### REFERENCE NUMERALS IN THE DRAWINGS

100 disclosed system in general
200 base tray
300 lid
400 barrier film
500 medical device

### DETAILED DESCRIPTION OF EMBODIMENTS OF THE INVENTION

The following detailed description is directed to certain specific embodiments of the invention. However, the invention can be embodied in a multitude of different ways as defined and covered by the claims and their equivalents. In this description, reference is made to the drawings wherein like parts are designated with like numerals throughout.

Unless otherwise noted in this specification or in the claims, all of the terms used in the specification and the claims will have the meanings normally ascribed to these terms by workers in the art.

Unless the context clearly requires otherwise, throughout the description and the claims, the words “comprise,” “comprising” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “above,” “below,” and words of similar import, when used in this application, shall refer to this application as a whole and not to any particular portions of this application.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform routines having steps in a different order. The teachings of the invention provided herein can be applied to other systems, not only the systems described herein. The various embodiments described herein can be combined to provide further embodiments. These and other changes can be made to the invention in light of the detailed description.

Any and all the above references and U.S. patents and applications are incorporated herein by reference. Aspects of the invention can be modified, if necessary, to employ the systems, functions and concepts of the various patents and applications described above to provide yet further embodiments of the invention.

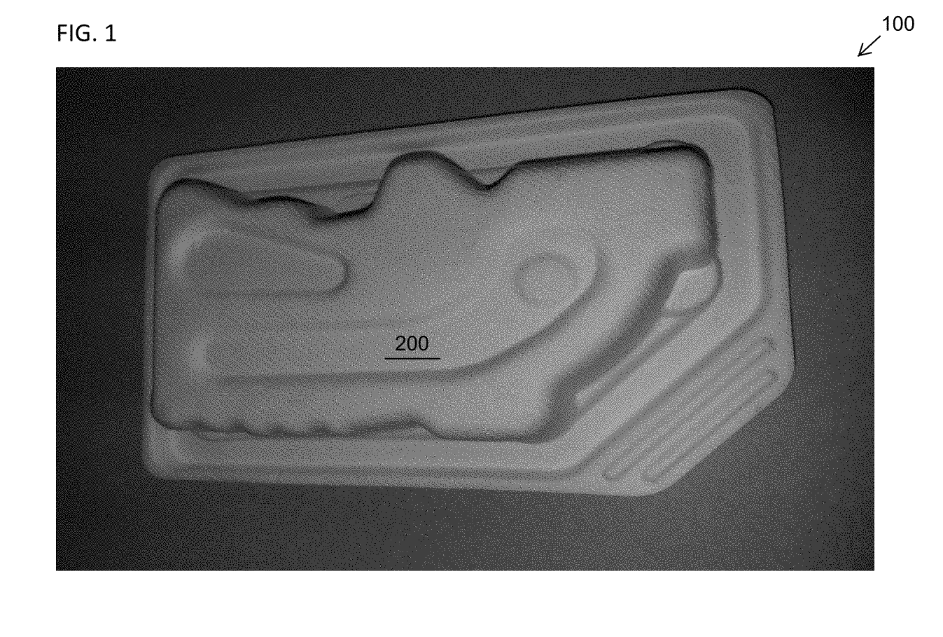

FIG. 1 depicts a disclosed base in general. The base may be formed to comport with a medical device or surgical instrument. The base may be made of plant based materials. The base may be made with sufficient curvatures and rigidity to protect the contents from damage upon dropping or other foreseeable impacts.

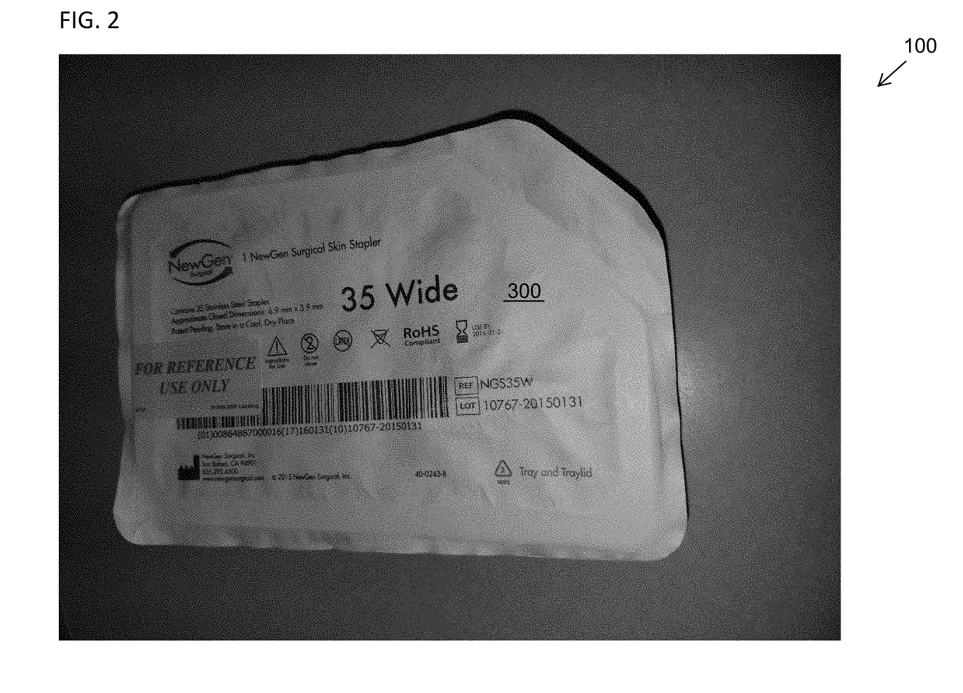

FIG. 2 depicts a disclosed lid or cover system. The lid or cover system may have coatings or other means to present a microbial barrier to keep the retained surgical instrument sterile. The lid or cover system may be made from plant based materials or from more traditional materials such as either paper or Tyvek.

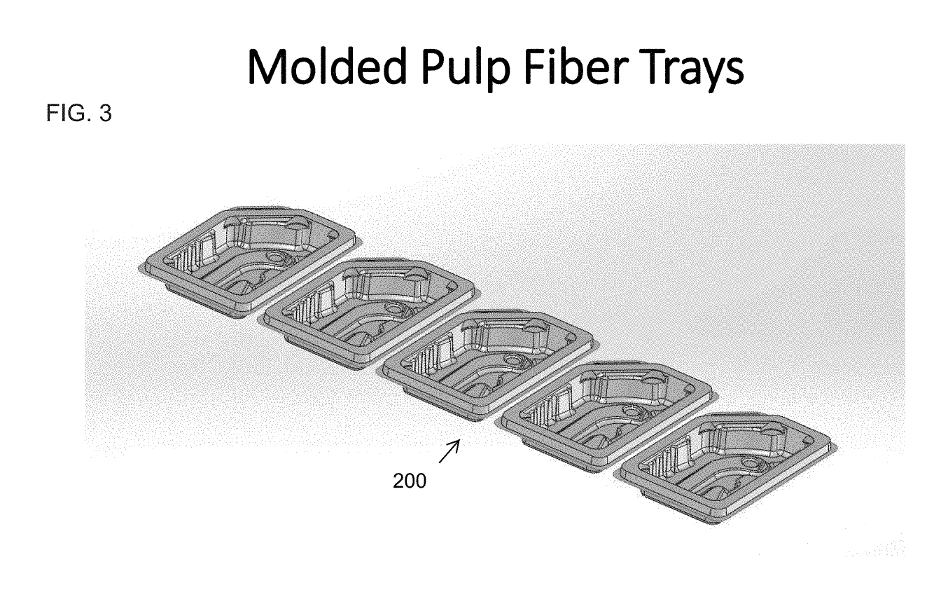

FIG. 3 depicts a perspective view of molded pulp fiber trays.

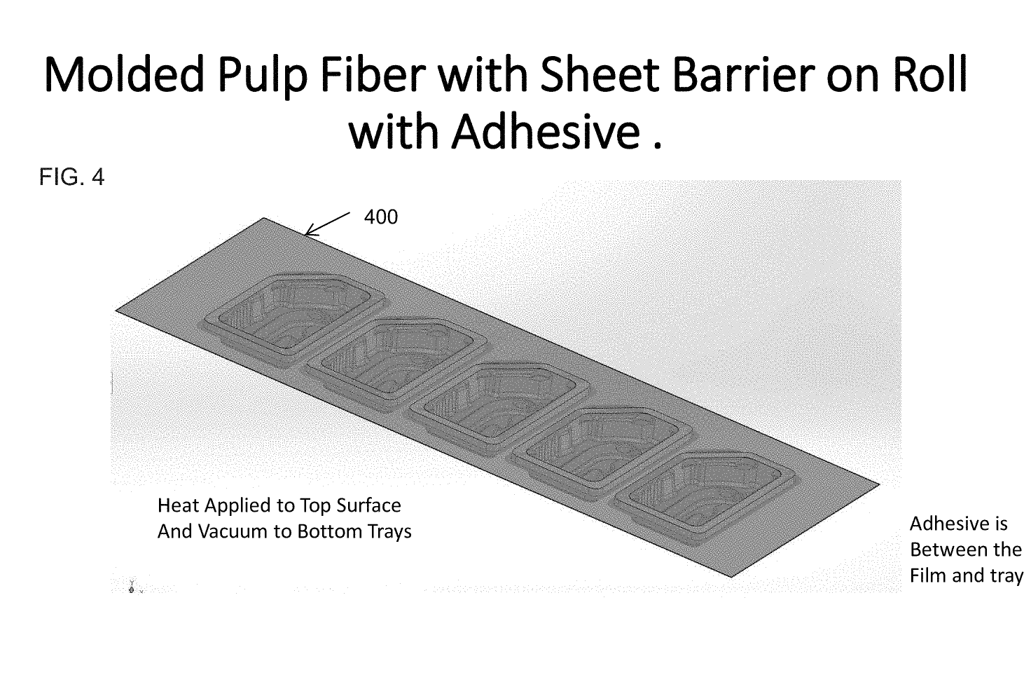

FIG. 4 depicts Molded Pulp Fiber (MPF) with sheet barrier on roll with adhesive.

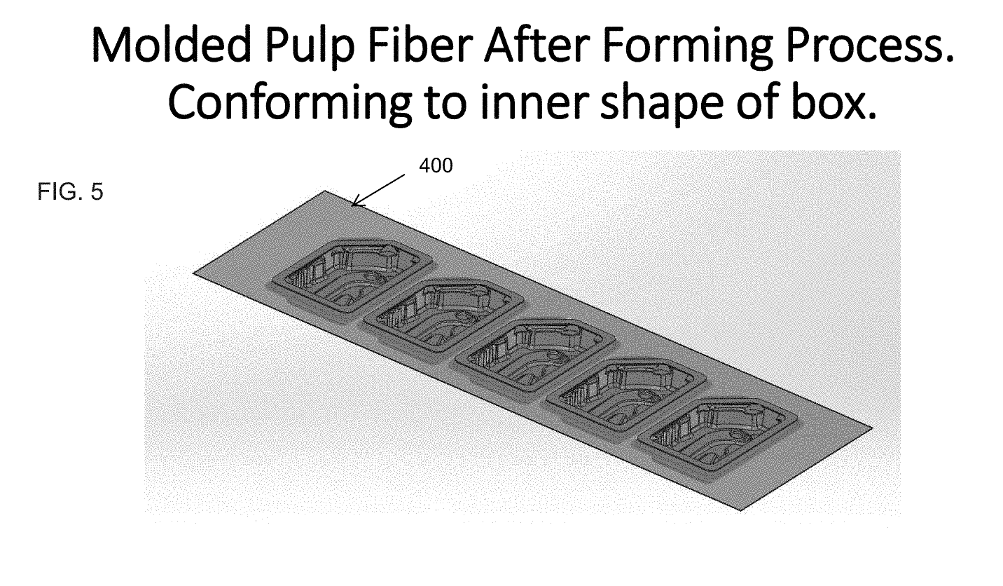

FIG. 5 depicts MPF after a forming process. The result conforms to the inner shape of a box.

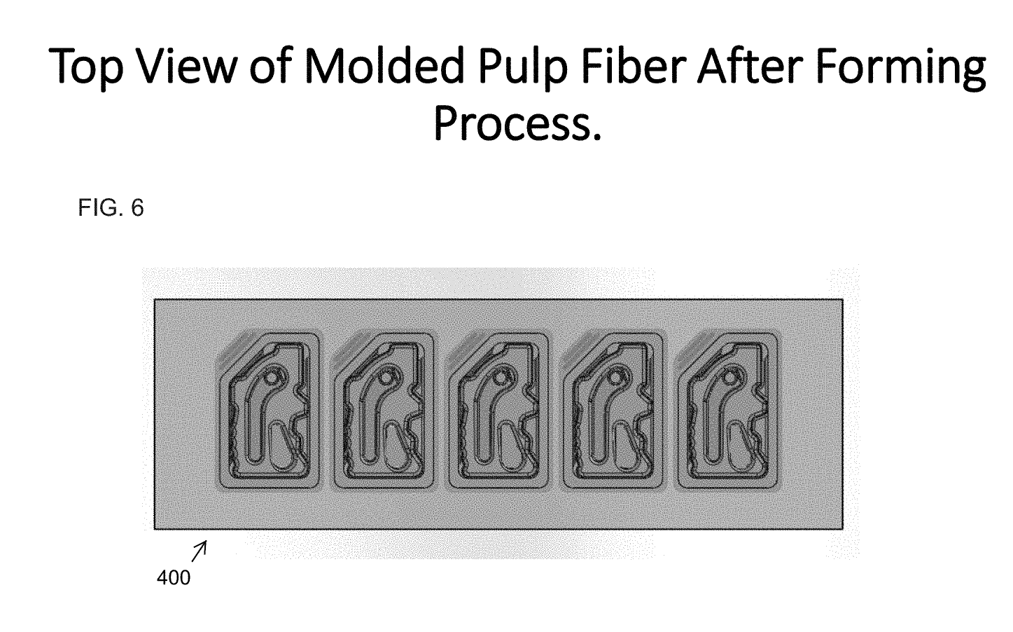

FIG. 6 depicts a top view of a MPF after a forming process.

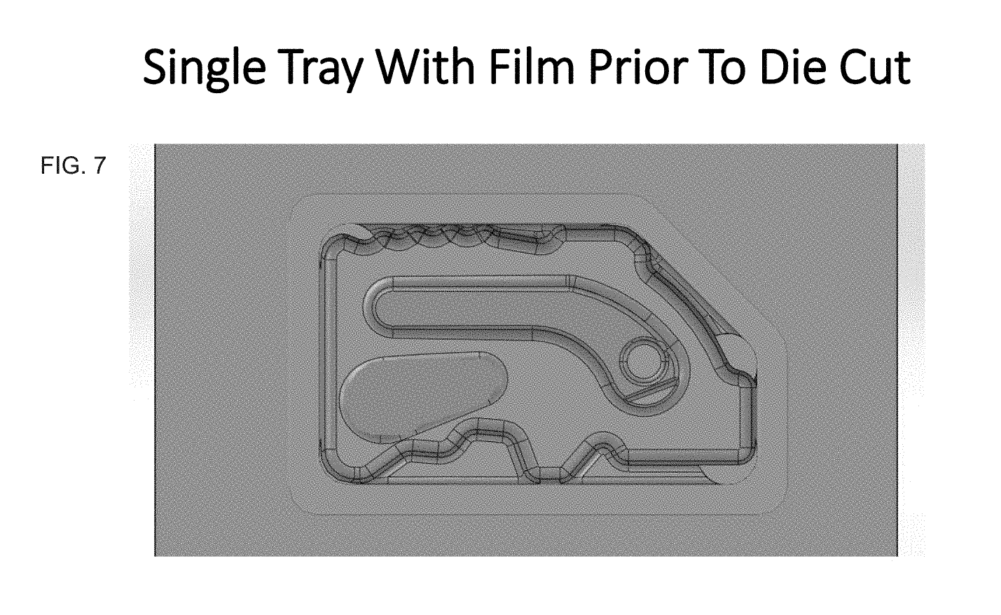

FIG. 7 depicts a single tray with film prior to being cut with a die.

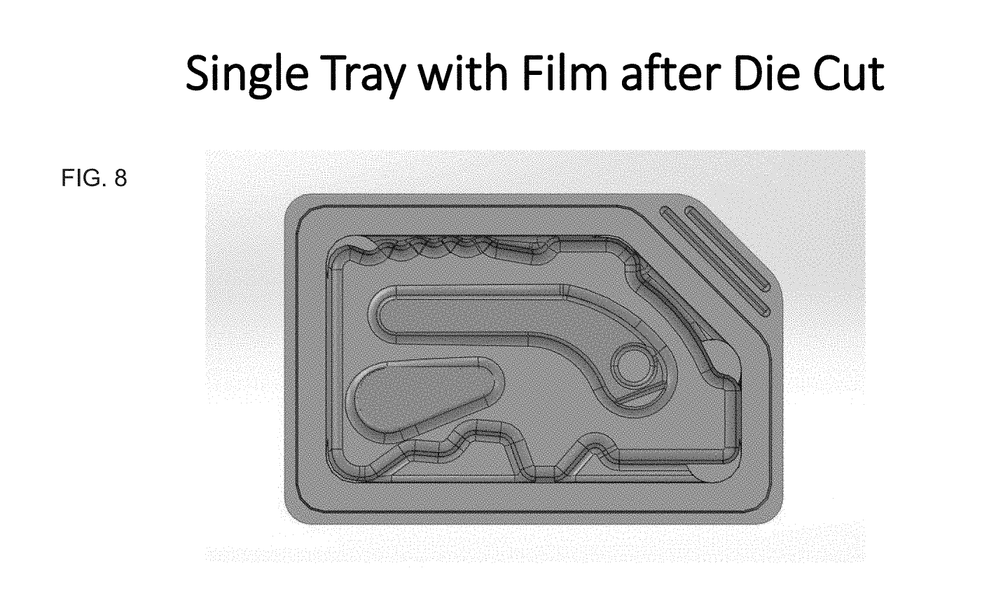

FIG. 8 depicts a single tray with a film after being cut with a die.

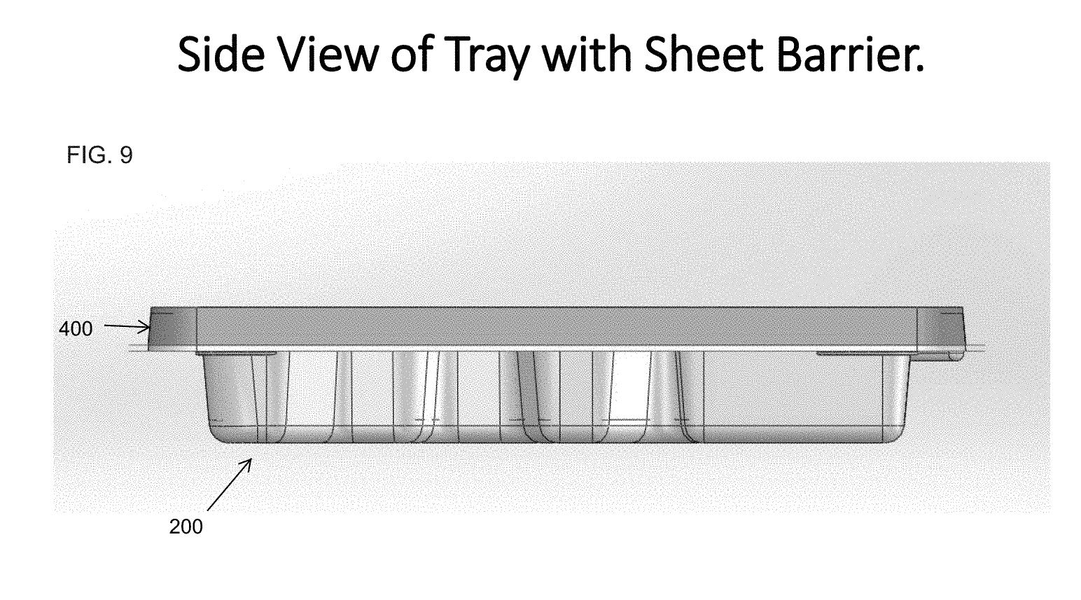

FIG. 9 depicts a side view of a tray with a sheet barrier.

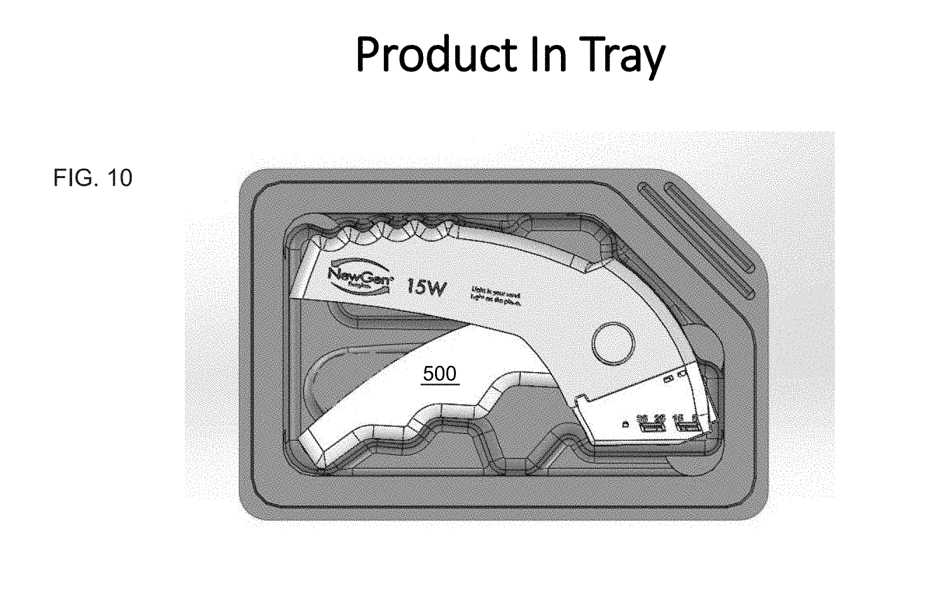

FIG. 10 depicts a product in a disclosed tray.

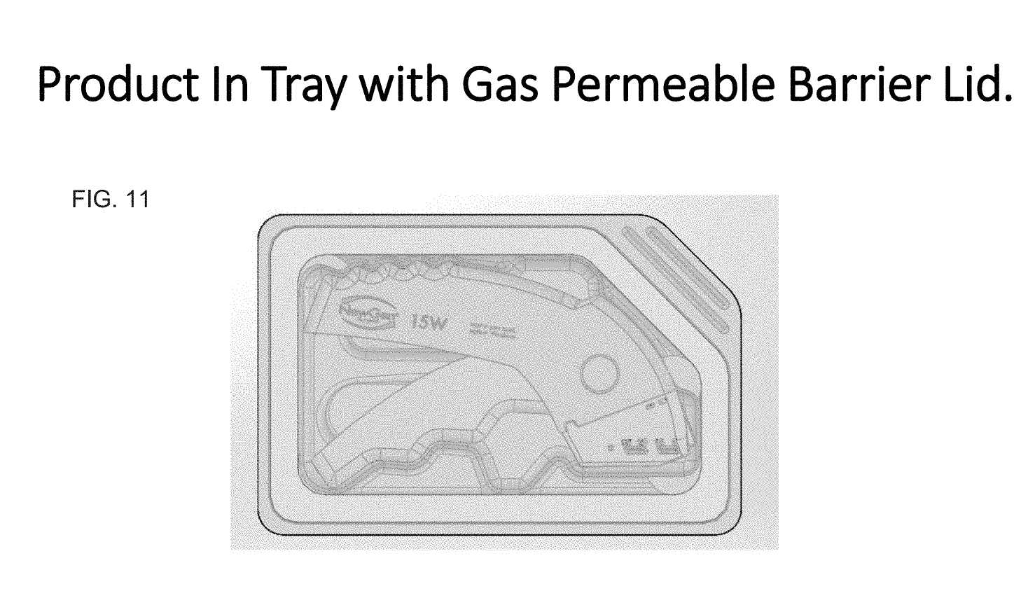

FIG. 11 depicts a medical product in disclosed tray with a gas permeable barrier lid.

The disclosed embodiments may include the following items.

Item 1. A packaging system with a microbial barrier, the system comprising:

a) a base tray comprised of plant based material, the base tray comprising a bottom and four sides, with the bottom and the four sides defining a cavity void suitable to contain a medical instrument;

b) the base tray having an inside surface and an outside surface with the inside or outside surface coated with a film, the film being comprised of a plant based product, the film functioning as a microbial barrier preventing bacteria from reaching the cavity void;

c) a lid sealed to the top surfaces of the base tray, the lid functioning to prevent bacteria from reaching the cavity void.

Item 2. The system of item 1 wherein the lid is made of paper.

Item 3. The system of item 1 wherein the lid is made of Tyvek.

Item 4. The system of item 1 wherein the lid is comprised of a gas permeable barrier.

Item 5. The system of item 1 wherein the outside surface of the base tray, instead of the inside surface of the base tray, is coated with the film.

Item 6. The system of item 1 wherein the film is comprised of a petroleum based product.

What is claimed is:

1. A packaging system with a microbial barrier, the system comprising:
a) a base tray comprised of plant based material, the base tray comprising a bottom and four sides, with the bottom and the four sides defining a cavity void suitable to contain a medical instrument;
b) the base tray having an inside surface and an outside surface with the inside or outside surface coated with a film, the film being comprised of a plant based product, the film functioning as a microbial barrier preventing bacteria from reaching the cavity void;
c) a lid sealed to the top surfaces of the base tray, the lid functioning to prevent bacteria from reaching the cavity void.

2. The system of claim 1 wherein the lid is made of paper.

3. The system of claim 1 wherein the lid is made of Tyvek.

4. The system of claim 1 wherein the lid is comprised of a gas permeable barrier.

5. The system of claim 1 wherein the outside surface of the base tray, instead of the inside surface of the base tray, is coated with the film.

6. The system of claim 1 wherein the film is comprised of a petroleum based product.
